(19) United States
(12) Patent Application Publication
(10) Pub. No.: US 2024/0138587 A1
(43) Pub. Date: May 2, 2024

(54) INDIVIDUAL IDENTIFICATION AND CUSTOMIZED ADJUSTMENT BY A SMART BED

(71) Applicant: Purple Innovation, LLC, Lehi, UT (US)

(72) Inventors: Thomas Andrew Bennett, Irvine, CA (US); James Grutta, Draper, UT (US)

(21) Appl. No.: 17/978,186

(22) Filed: Oct. 31, 2022

(51) Int. Cl. A47C 31/12; A47C 21/04; A61M 21/02

(52) U.S. Cl. CPC A47C 31/123; A47C 21/042; A47C 21/048; A61M 21/02; A61M 2021/0066; A61M 2205/3303

(57) ABSTRACT

A smart bed that can identify one or more individuals on the sleep surface of a mattress of the smart bed. One or more features of the smart bed may be actuated or adjusted based on the identity of the individual on the sleep surface. Methods for identifying an individual on a sleep surface of a mattress of a smart bed and, optionally, actuating or adjusting at least one feature of the smart bed in response to the identity of the individual on the sleep surface are also disclosed.

### TECHNICAL FIELD

This disclosure relates generally to smart beds and, more specifically, to smart beds that can identify one or more individuals on the sleep surface of a mattress of the smart bed. Additionally, this disclosure relates to smart beds that automatically actuate or adjust one or more features based on the identity of the individual on the sleep surface. This disclosure also relates to methods for identifying an individual on a sleep surface of a mattress of a smart bed and, optionally, actuating or adjusting at least one feature of the smart bed in response to the identity of the individual on the sleep surface.

### SUMMARY

In various aspects, a smart bed that can identify, or recognize, an individual on a sleep surface of a mattress thereof is disclosed. As a limited number of people (e.g., one person, members of a couple, members of a family, etc.) typically use a smart bed, the individuals who may use the smart bed may be distinguished from each other based on information, or data, that may be readily obtained with sensors of the smart bed. Such data may include the respiratory rate, other aspects of respiration (e.g., changes in respiratory rate, respiratory patterns, snoring, etc.), heart rate, other aspects associated with heart rate (e.g., changes in heart rate, heart rate patterns, etc.), temperature, humidity, sleeping location, or resting position or sleeping position of an individual, or a combination of any of the foregoing. Once the smart bed has identified an individual, the smart bed may automatically adjust one or more features in a manner that is customized to the individual.

Such a smart bed may include a mattress that defines the sleep surface, at least one sensor associated with the sleep surface, and a processor that communicates with the at least one sensor and controls one or more features, or functions, of the mattress. A smart bed may also include a foundation for the mattress (e.g., an adjustable foundation, etc.).

The mattress may have any suitable configuration. As a few examples, the mattress may comprise a conventional mattress with coils, a so-called “memory foam” mattress, a mattress with one or more bladders that may be selectively pressurized, a gel mattress, or a mattress that includes a combination of these and/or other features.

The at least one sensor may be capable of detecting motion or another activity (e.g., proximity, the application of pressure, etc.) to a surrounding area of the sleep surface of the mattress. Each sensor may comprise an accelerometer. More specifically, the at least one sensor may be positioned adjacent to the sleep surface at a location where it may detect the presence of an individual on the sleep surface. The at least one sensor may detect a respiratory rate of an individual on the sleep surface, variations in respiration by the individual, a heart rate of an individual on the sleep surface, variations in the individual's heart rate, a temperature of at least one part of a body of an individual on the sleep surface, and/or a humidity generated by at least one part of a body of an individual on the sleep surface. The at least one sensor may even detect a position (e.g., a sleep position, etc.) of an individual on the sleep surface. In some embodiments, the at least one sensor may be positioned at a location where an individual would be expected to rest their legs when laying in a sleeping position on the sleep surface. In other embodiments, the at least one sensor may comprise a group of sensors, such as any combination of a sensor at the side of the mattress (e.g., a sensor positioned over a rail of the mattress, on a side of a rail of the mattress, within a rail of the mattress, etc.), a sensor at a location where an individual would be expected to rest their body when assuming a sleeping position on the sleep surface, a sensor at a location where the individual would be expected to rest their legs when assuming a sleeping position on the sleep surface, and a sensor at any other location that may enable it detect activities that are ordinarily associated with an individual “climbing into bed” (e.g., sitting or otherwise moving on a side of the mattress or on the mattress, moving toward a center of the mattress, and extending their legs, etc.).

The processor that communicates with (e.g., receives signals from, etc.) each sensor associated with the sleep surface of the mattress may comprise one or more processing elements of a suitable type. In some embodiments, the processor may comprise one or more microcontrollers, which may execute an embedded program (e.g., firmware) to control one or more dedicated functions. The processor may be carried by the mattress (e.g., internally, adjacent to a foot of the mattress; etc.).

Based on data generated by the at least one sensor, the processor may be programmed to detect a presence of an individual on the sleep surface of the mattress. Based on data generated by the at least one sensor, the processor may also be programmed to determine a location of the individual on the sleep surface. Based on data generated by the at least one sensor, the processor may be programmed to determine an orientation of the individual on the sleep surface (e.g., whether the individual is in an inclined position, or a sitting position, on the sleep surface; whether the individual is in a reclined position, or a sleeping position, on the sleep surface; etc.). The processor may be programmed to determine one or more of a respiratory rate of an individual on the sleep surface, variations in respiration of an individual on the sleep surface, a heart rate of an individual on the sleep surface, variations in the heart rate of an individual on the sleep surface, a temperature of a part of a body of an individual on the sleep surface, a humidity generated by a part of a body of an individual on the sleep surface, or another potentially identifying characteristic of an individual on the sleep surface. Programming of the processor may enable it to identify the individual who is present on the sleep surface; a processor programmed in this manner may actuate the at least one feature in a manner that corresponds to an identity of the individual present on the sleep surface.

Once the processor determines that an individual is on the sleep surface of the mattress, that the individual has likely assumed a resting position or sleeping position on the sleep surface, and identified the individual, the processor may execute (e.g., activate, adjust, etc.) one or more features of the smart bed. The one or more features may be customized to the individual who has been identified by the processor. A few nonlimiting examples of features that may be executed include adjusting an orientation of the mattress or a portion thereof, adjusting a firmness (or softness) of the mattress or a portion thereof (e.g., pressurization of a bladder within the mattress, etc.), ventilating the mattress or a portion thereof, adjusting a temperature of the mattress or a portion thereof, actuating an anti-snore feature of the smart bed (e.g., its foundation, its mattress, etc.), etc.

The settings for one or more features of the smart bed, which may dictate any adjustments to the smart bed upon identifying an individual upon the smart bed, may be stored. For example, the settings may be stored by memory of the smart bed. The settings stored by memory of the smart bed may be accessed by the processor of the smart bed, which may, if necessary, adjust the one or more features of the smart bed.

Alternatively, the settings may be stored by memory of a portable electronic device (e.g., a smart phone, a tablet computer, a smart watch, etc.) used by the individual who has been identified by the smart bed. Upon identifying the individual, the processor of the smart bed may wake the processor of the portable electronic device used by the individual. The processor of the portable electronic device, under control of a smart bed control app, may cause a transceiver of the portable electronic device to communicate appropriate settings to the transceiver and processor of the smart bed through antennas associated with the transceivers. The processor of the smart bed may then adjust the one or more features of the smart bed, if necessary.

The individual-specific settings for one or more features of the smart bed may be based on a predetermined preference of the individual on the mattress (e.g., as programmed by the individual or another person) or a predetermined sleep condition of the individual (e.g., as “learned” by a processor and programming of the smart bed or a portable electronic device used by the individual, etc.).

In a method of this disclosure, as an individual climbs onto a sleep surface of a mattress of a smart bed, the smart bed (i.e., sensors and a processor of the smart bed) may detect a presence of the individual on the sleep surface, determine a location of the individual on the sleep surface and determine one or more potentially identifying characteristics of the individual (e.g., respiratory rate, variations in respiratory rate, heart rate, variations in heart rate, body temperature, humidity generated, resting position, sleeping position, etc.). With such information, the smart bed may attempt to identify, or recognize, the individual.

Upon identifying the individual, the smart bed may make one or more adjustments based on the individual's identity. Each adjustment may be based on settings stored by memory of the smart bed and/or memory of a portable electronic device used by the individual who has been identified on the sleep surface of the mattress. Where the settings are stored by memory of a portable electronic device, the smart bed (e.g., through its processor and a transceiver and its associated antenna) may establish communication with the portable electronic device. The settings may comprise at least one predetermined preference of the individual on the sleep surface or at least one predetermined sleep condition of the individual (e.g., as identified by the smart bed, etc.). Examples of adjustments that may be made include, without limitation, adjusting an orientation of the mattress or a portion thereof, adjusting a firmness (or softness) of the mattress or a portion thereof (e.g., pressurization of a bladder within the mattress, etc.), ventilating the mattress or a portion thereof, adjusting a temperature of the mattress or a portion thereof, and actuating an anti-snore feature of the smart bed (e.g., its foundation, its mattress, etc.).

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 provides a representation of an embodiment of a smart bed according to this disclosure;

FIG. 2 provides a top view of the embodiment of the smart bed shown in FIG. 1;

FIG. 3 is a side view of the embodiment of the smart bed shown in FIG. 1;

FIG. 4 is a foot end view of the embodiment of the smart bed shown in FIG. 1;

FIG. 5 is a flow diagram illustrating various acts that may occur in an embodiment of a method for identifying an individual on a sleep surface of the mattress of the smart bed and actuating one or more features and/or making one or more adjustments to the smart bed based on the identity of the individual on the sleep surface; and

FIGS. 6 and 7 provide top views of the embodiment of the smart bed shown in FIG. 1, illustrating a sequence of events that may occur as an individual assumes a resting position or a sleeping position on the sleep surface.

### DETAILED DESCRIPTION

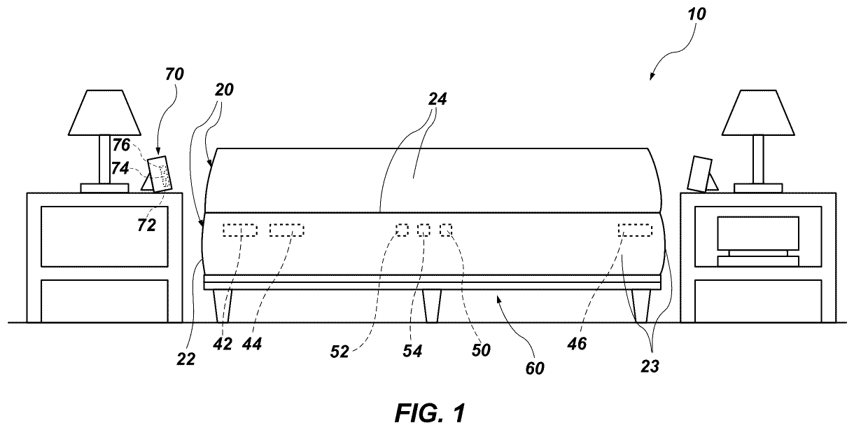

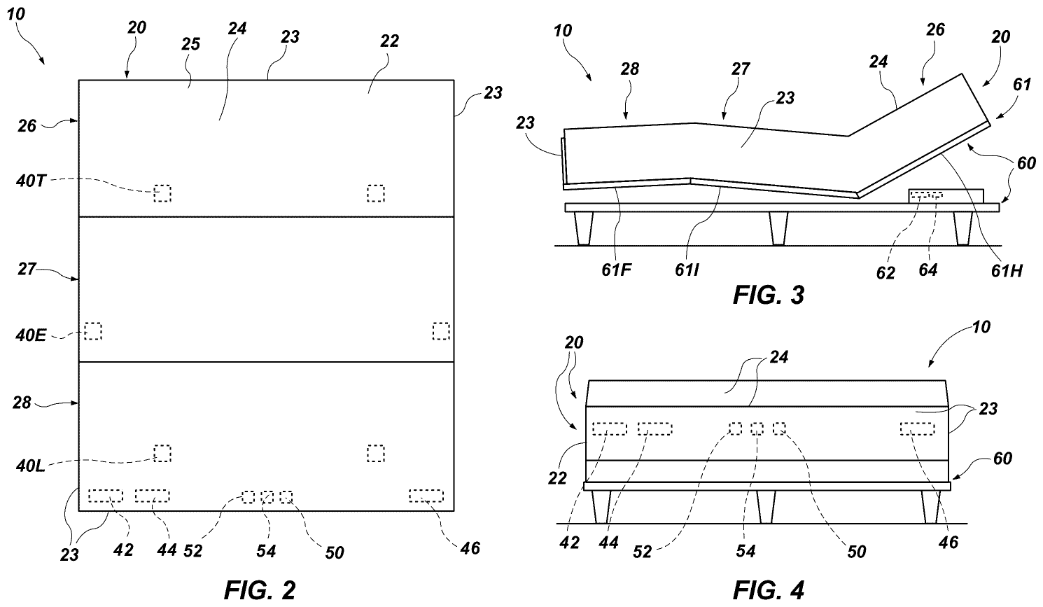

FIGS. 1-4 illustrate an embodiment of a smart bed 10. The smart bed 10 includes a mattress 20, at least one sensor 40 (including, without limitation, one or more thoracic sensors 40T, one or more leg sensors 40L, and/or one or more ambient environment sensors 40E, etc.), a processor 50, at least one transceiver 52 (along with an associated antenna), and memory 54. In addition, the smart bed 10 may include a foundation 60.

The mattress 20 of the smart bed 10 may have any suitable shape and dimensions and may include a cover 22 that contains various internal elements (not shown) of the mattress 20. The mattress may include edges 23, a top surface, or sleep surface 24, between top ends of the edges 23, and a bottom surface, or base 29, between bottom edges of the edges 23. The sleep surface 24 of the mattress 20 may be configured to be slept on by at least one individual. The sleep surface 24 may include edges 25 at locations adjacent to side edges 23 of the mattress 20, a head portion 26, an intermediate portion 27, and a foot portion 28.

The mattress 20 may comprise any suitable type of mattress, such as a conventional mattress with springs and foam, a foam mattress, a memory foam mattress, a mattress with pressurizable bladders (e.g., air chambers, etc.), a mattress that includes a gel grid (e.g., those available from Purple Innovation, LLC, of Lehi, Utah and disclosed by U.S. Pat. Nos. 7,060,213, 7,076,822, and 8,919,750, which may be formed from any suitable material, including, but not limited to, an extended A-B-A triblock copolymer, such as those disclosed by U.S. Pat. Nos. 6,413,458, 6,797,765 and 7,964,664; the entire disclosures of each of the foregoing patents are hereby incorporated herein), or a mattress that includes any combination of these and/or other features.

The one or more sensors 40 may be carried by the mattress 20. Without limitation, at least one sensor 40 may be positioned adjacent to the sleep surface 24 of the mattress 20. For example, a sensor 40 may be positioned within a cover 22 of the mattress 20 in a manner that enables the sensor 40 to detect activity occurring on a sleep surface 24 of the mattress. More specifically, each sensor 40 may be positioned within a recess or a receptacle that opens to and communicates with an outer surface of a component of the mattress 20 that carries the sensor 40, with the cover 22 being placed over the recess or receptacle. In other embodiments, the sensor 40 may be incorporated into the cover 22 or positioned between the cover 22 and internal components (not shown) (e.g., a top layer of foam, gel, etc.) of the mattress 20.

In some embodiments, the mattress 20 may carry a plurality of sensors 40. Without limitation, at least one thoracic sensor 40T may be positioned at the head portion 26 and/or the intermediate portion 27 of the mattress 20. At least one leg sensor 40L may be positioned at the foot portion 28 of the mattress 20. At least one ambient environment sensor 40E may be positioned at an edge 23 (e.g., on, adjacent to, or within a rail, etc.) of the mattress 20. In a specific embodiment, the mattress 20 may comprise a gel grid, with each thoracic sensor 40T and leg sensor 40L being positioned within a hollow column of the gel grid, adjacent to an upper surface of the gel grid, and each ambient environment sensor 40E being positioned within a recess or cavity that opens to or otherwise communicates with an outer surface of a side rail of the mattress 20.

The at least one thoracic sensor 40T may facilitate detection of the presence (or absence) of an individual on the sleep surface 24 of the mattress 20. In addition, the at least one thoracic sensor 40T may be used to monitor one or more of the individual's movement (e.g., motion, position on the sleep surface 24, etc.), respiration, and heart rate. The at least one thoracic sensor 40T may also monitor the temperature of the individual's body and, optionally, humidity (e.g., humidity at least partially generated by a part of the individual's body, etc.). In a specific embodiment, the at least one thoracic sensor 40T may include an inclinometer (e.g., an inclinometer available from STMicroelectronics of Plan-les-Ouates, Switzerland, etc.), an accelerometer (e.g., an accelerometer available from MEMSIC Inc. of Andover, Massachusetts, etc.), and a plurality of temperature/humidity sensors (e.g., a temperature and/or humidity sensor available from Sensiron AG of Stafa, Switzerland, etc.).

The at least one leg sensor 40L may facilitate the detection of the presence (or absence) of an individual on the sleep surface 24 of the mattress 20. In addition, the at least one leg sensor 40L may be used to monitor the individual's movement (e.g., motion, position, etc.), respiration, and heart rate. The at least one leg sensor 40L may monitor the temperature of the individual's legs and, optionally, humidity. In a specific embodiment, the at least one leg sensor 40L may include an accelerometer (e.g., an accelerometer available from MEMSIC Inc. of Andover, Massachusetts, etc.) and a plurality of temperature/humidity sensors (e.g., a temperature and/or humidity sensor available from Sensiron AG of Stafa, Switzerland, etc.).

The at least one ambient environment sensor 40E may facilitate the detection of the presence (or absence) of an individual in proximity to a side edge 23 of the mattress 20 or on the sleep surface 24 of the mattress 20. In addition, the at least one ambient environment sensor 40E may be used to monitor information about an environment (e.g., a room, etc.) within which the mattress 20 is located, such as its temperature and/or humidity. In a specific embodiment, the at least one ambient environment sensor 40E may include an accelerometer (e.g., an accelerometer available from MEMSIC Inc. of Andover, Massachusetts, etc.) and a temperature/humidity sensor (e.g., a temperature and/or humidity sensor available from Sensiron AG of Stafa, Switzerland, etc.).

In the illustrated embodiment of mattress 20, each thoracic sensor 40T and leg sensor 40L is shown at a location where an individual is expected to lie on the sleep surface 24 of the mattress 20 as he or she sleeps, with the mattress 20 having a size that accommodates two individuals. In other embodiments, including small mattresses (e.g., twin size, twin XL (extra long) size, full size, etc.) and large mattresses (e.g., queen size, king sizes, etc.), thoracic sensors 40T and leg sensors 40L may be positioned along a midline of the mattress-alone or in combination with other sensor placements.

The mattress 20 may also include one or more other features. Examples of such features include, without limitation, a pressurization system 42, a ventilation system 44, and a temperature control system 46. Embodiments of a pressurization system 42, which may facilitate adjustment of a pressure (e.g., air pressure, etc.) of one or more portions of the mattress 20 or the entire mattress 20, are disclosed by U.S. Pat. No. 11,213,139 and U.S. Patent Application Publication US 2022/0273113 A1, the entire disclosures of which are hereby incorporated herein. Embodiments of a ventilation system 44, which may ventilate part or all of the mattress 20, are disclosed by U.S. Pat. No. 11,311,111, the entire disclosure of which is hereby incorporated herein.

The processor 50 of the smart bed 10 may comprise one or more processing elements of a suitable type (e.g., microprocessors, microcontrollers, digital signal processors (DSPs), etc.). In some embodiments, the processor 50 may comprise one or more microcontrollers, which may execute embedded programming (e.g., firmware) to control one or more dedicated functions. The processor 50 may be carried by the mattress 20 of the smart bed 10, by the foundation 60 of the smart bed 10, or elsewhere.

Activity detected by the sensors 40 may be communicated to the processor 50. The processor 50 may execute a program to determine whether the data from the sensors 40 indicates that an individual is present on the sleep surface 24 of the mattress 20. The program may also enable the processor to generate data regarding the individual, or “individual data,” such as the respiratory rate of the individual, other aspects of the individual's respiration (e.g., changes in respiratory rate, respiratory patterns, snoring, etc.), the heart rate of the individual, other aspects of the individual's heart rate (e.g., changes in heart rate, patterns in the individual's heart rate, etc.) a temperature of part of the individual's body, a humidity generated by part of the individual's body, and a sleep position assumed by the individual on the sleep surface 24.

If the processor 50 determines that an individual has assumed a sleeping position on the sleep surface 24, determines that a sequence of events has occurred that culminated in the individual assuming a resting position or a sleeping position on the sleep surface 24, identifies the individual on the sleep surface 24, or the like, then the processor 50 may execute one or more programs that affect a state of the mattress 20 and/or the smart bed 10 of which the mattress 20 is a part.

The transceiver 52 of the smart bed 10 may be associated with the processor 50 of the smart bed 10 in a manner that enables the processor 50 to communicate with a transceiver 74 associated with a processor 72 of a portable electronic device 70 (e.g., a smart phone, a tablet computer, a smart watch, etc.) or another device. Without limitation, the transceiver 52 may comprise one or more of a WiFi transceiver, a Bluetooth® transceiver, a near-field communication (NFC) (e.g., radiofrequency (RF), etc.) transceiver, a 2.4 GHz RF transceiver, or the like, along with an associated antenna. The transceiver 52 may be carried by the mattress 20 of the smart bed, the foundation 60 of the smart bed 10, or elsewhere. In some embodiments, the transceiver 52 may be associated with (e.g., located adjacent to, packaged with, etc.) the processor 50 of the smart bed 10.

The memory 54 of the smart bed 10 may comprise any suitable type of computer readable memory accessible by the processor 50 of the smart bed 10. Without limitation, the memory 54 may comprise primary memory (e.g., RAM (random access memory), ROM (read-only memory), etc.), secondary memory (e.g., a flash-based memory, which may be in the form of a solid state drive (SSD), etc.; etc.). The memory 54 may be carried by the mattress 20 of the smart bed, the foundation 60 of the smart bed 10, or elsewhere. In some embodiments, the memory 54 may be associated with (e.g., located adjacent to, packaged with, etc.) the processor 50 of the smart bed 10.

The foundation 60 of the smart bed 10 may include any suitable foundation for the mattress 20. In some embodiments, the foundation 60 may comprise an adjustable platform (e.g., the Purple® Ascent Base™ adjustable platform, available from Purple Innovation, LLC, of Lehi, Utah, etc.). Such a foundation 60 may include a processor 62, which may control movement of one or more adjustable sections 61H, 61I, 61F of a platform 61 that carries the mattress 20. In addition, such a foundation 60 may include a transceiver 64 (e.g., a WiFi transceiver, a Bluetooth® transceiver, a near-field communication (NFC) (e.g., RF, etc.) transceiver, a 2.4 GHz RF transceiver, etc.), along with an associated antenna. The transceiver 64 may receive signals from the processor 50 of the smart bed 10 and/or a processor 72 of a portable electronic device 70 that may cause the processor 62 of the foundation 60 to move one or more sections 61A, 61I, 61F of the platform 61 of the foundation 60 in a desired manner (e.g., to move the mattress 20 or a portion thereof to a desired orientation, to operate an anti-snore feature of the foundation 60, etc.). In other embodiments, the foundation 60 may comprise a stationary foundation (e.g., a platform style bed frame, a conventional bed frame and box spring, etc.).

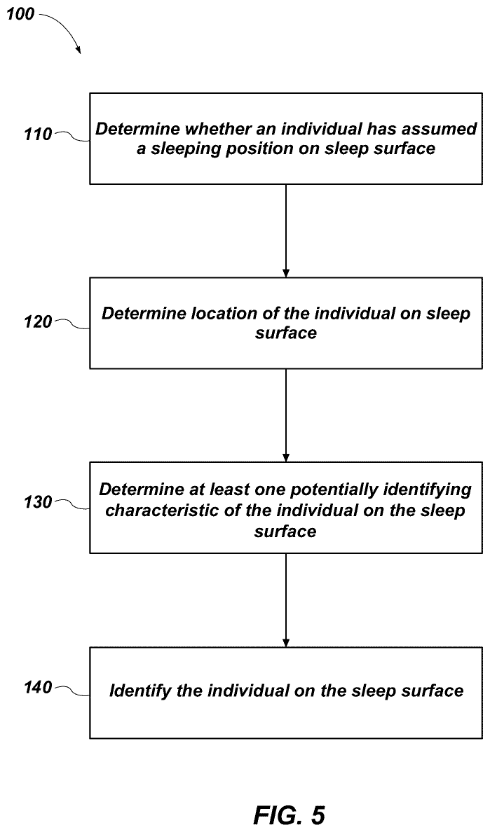

FIG. 5 is a diagram illustrating the flow of an embodiment of a method 100 of adjusting a bed based on an identity of an individual on a sleep surface 24 of a mattress 20 of a smart bed 10, such as that depicted by FIGS. 1-4. The method 100 may include detecting the presence of an individual on the sleep surface 24, at reference 110, determining a location of the individual on the sleep surface 24, at reference 120, and determining at least potentially identifying characteristic of the individual on the sleep surface 24 (e.g., a respiratory rate of the individual, other aspects of the individual's respiration, a heart rate of the individual, other aspects of the individual's heart rate, a temperature of a part of a body of the individual, a humidity generated by a part of a body of the individual, a sleep position of the individual, etc.), at reference 130. Based on any of the individual information collected at reference 130 or on a combination of such information (i.e., two or more potentially identifying characteristics), the method 100 may include identifying the individual on the sleep surface 24, at reference 140.

Once the individual on the sleep surface 24 has been identified, the method 100 may, at reference 150, include actuating or adjusting one or more features of the smart bed 10 in a manner that customizes, or tailors, the smart bed 10 or a portion thereof for the individual who has been identified. A few nonlimiting examples of features that may be actuated or adjusted include adjusting an orientation of the mattress or a portion thereof, adjusting a firmness (or softness) of the mattress or a portion thereof (e.g., pressurization of a bladder within the mattress, etc.), ventilating the mattress or a portion thereof, adjusting a temperature of the mattress or a portion thereof, actuating an anti-snore feature of the smart bed (e.g., its foundation, its mattress, etc.), etc.

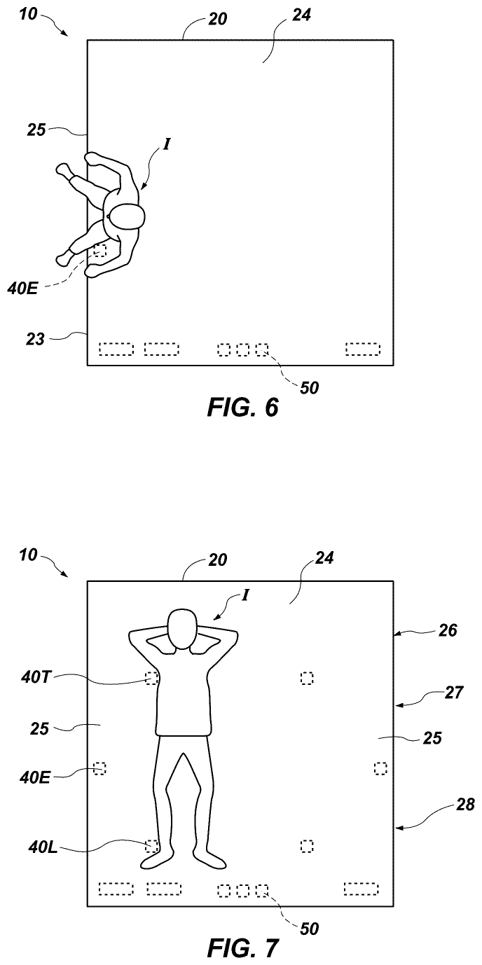

With reference to FIGS. 6 and 7, an embodiment of an event or a sequence of events that may enable the processor 50 to identify an individual I on the sleep surface 24 of the mattress 20 of the smart bed 10 is illustrated. The event may comprise detection of an individual I in a resting position or a sleeping position on the sleep surface 24. The sequence of events may comprise a sequence of events that ordinarily occurs when an individual I “gets into bed” or “climbs into bed” or a sequence of events that occurs as a particular individual I (e.g., an individual I who typically uses the smart bed, etc.) assumes a resting position or a sleeping position on the sleep surface 24.

As illustrated by FIG. 6, an ambient environment sensor 40E at the edge 23 of the mattress 20 may detect an individual I approaching and/or sitting or otherwise positioning themselves on the edge 25 of the sleep surface 24 of the mattress 20. As the ambient environment sensor 40E detects such activity, it may “wake” the processor 50 by communicating data regarding such activity to the processor 50. Then, as shown in FIG. 7, a sensor 40T at a location where an individual I would be expected to rest their body (e.g., the head portion 26 of the mattress 20, the intermediate portion 27 of the mattress 20, etc.) and/or a sensor 40L at a location where the individual I would be expected to rest their legs (e.g., the foot portion 28 of the mattress 20, etc.) may detect the individual I assuming a resting position or sleeping position on the sleep surface 40. Alternatively or additionally, one or more sensors 40E, 40T, 40L may detect other activity associated with climbing into bed, such as movement from the edge 25 of the sleep surface 24 towards a center of the sleep surface 24, movement in a manner that resembles extension of an individual I's legs toward the foot portion 28 of the sleep surface 24, and the like.

With returned reference to FIGS. 1-4, upon determining that an individual has assumed a resting position or a sleeping position on the sleep surface 24 of the mattress 20 of the smart bed 10 (at reference 110 of FIG. 5), optionally determining a location of the individual on the sleep surface (at reference 120 of FIG. 5; e.g., left side or right side of a queen size or larger bed, etc.) and identifying the individual on the sleep surface 24 (at reference 140 of FIG. 5), the processor 50 may actuate or adjust one or more features of the smart bed 10 to customize at least a portion of the mattress 20 for the identified individual. In some embodiments, the processor 50 may actuate or adjust other features of the smart bed 10 based on the identity of the individual on the sleep surface 24. As a few examples, the processor 50 may: provide the mattress 20 with a pre-set firmness with the a pressurization system 42 of the mattress 20 (see, e.g., U.S. Pat. No. 11,213,139); cause the sleep surface 24 to be ventilated by a ventilation system 44 of the mattress 20 (see, e.g., U.S. Pat. No. 11,311,111); cause the sleep surface 24 to be heated or cooled to a pre-set temperature; adjust an orientation of at least a portion of the mattress 20 (e.g., with the platform 60, etc.); turn on an anti-snore feature of the smart bed 10 (e.g., of its foundation 60, its mattress 20, etc.); or adjust any other feature of the smart bed 10 based on the identity of the individual on the sleep surface 24 of the mattress 20.

Each feature of the smart bed 10 that is actuated and/or adjusted and the manner in which the feature is actuated or adjusted may be based on a predetermined preference of the individual on the sleep surface 24 or at least one predetermined sleep condition of the individual (e.g., as identified by the smart bed 10, etc.).

In some embodiments, the processor 50 of the smart bed 10 may access the memory 54 of the smart bed 10 to actuate and/or adjust one or more features of the smart bed 10 based on the identity of the individual. In other embodiments, the processor 50 may receive settings for the identified individual from the portable electronic device 70 associated with the individual.

In embodiments where the processor 50 of the smart bed 10 communicates with the portable electronic device 70 of the individual who has been identified on the sleep surface 24 of the mattress 20, the processor 50 may be programmed to wake a processor 72 of a portable electronic device 70 (e.g., a smart phone, a tablet computer, a smart watch, etc.) associated with the identified individual. This may be accomplished by way of the transceiver 52 of the smart bed 10 and a transceiver 74 (along with an associated antenna) of the portable electronic device 70. The processor 50 of the smart bed 10 may cause the processor 72 of the portable electronic device 70 to execute a smart bed control app stored by memory 76 of the portable electronic device 70. The smart bed control app may be used to store settings specific for the individual and, in some embodiments, modify settings and/or generate new settings in response to how the individual sleeps (e.g., the individual's sleep position, movement while sleeping (which may be correlated with the time(s) of movement), movement of bedding while sleeping (which may be correlated with time(s) of movement), snoring, etc.). Stated another way, the smart bed control app may “learn” about how the individual sleeps and modify or generated settings based on the information it learns. The smart bed control app may cause the processor 72 to retrieve appropriate settings from memory 76 and transmit the settings to the processor 50 of the smart bed 10 (e.g., by way of the transceiver 74 of the portable electronic device 70 to the transceiver 52 of the smart bed 10, etc.) through antennas associated with the transceivers 52 and 74. The processor 50 of the smart bed 10 may then use the settings to actuate or adjust one or more features of the smart bed 10 based on the identity of the individual on the sleep surface 24 of the mattress 20.

Although this disclosure provides many specifics, these should not be construed as limiting the scope of any of the claims that follow, but merely as providing illustrations of some embodiments of elements and features of the disclosed subject matter. Other embodiments of the disclosed subject matter, and of their elements and features, may be devised which do not depart from the spirit or scope of any of the claims. Features from different embodiments may be employed in combination. Accordingly, the scope of each claim is limited only by its plain language and the legal equivalents thereto.

What is claimed:

1. A smart bed, comprising:
a mattress including at least one sensor; and
a processor that communicates with the at least one sensor, the processor programmed to process data from the at least one sensor to identify the individual on the mattress.

2. The smart bed of claim 1, wherein the processor is programmed to determine individual data comprising a respiratory rate, a heart rate, a temperature, a humidity, a sleeping location, and/or a sleeping position of the individual.

3. The smart bed of claim 2, wherein the processor is further programmed to identify the individual on the mattress from the individual data.

4. The smart bed of claim 3, wherein the processor is programmed to identify the individual based on a combination of the individual data.

5. The smart bed of claim 1, wherein the processor is further programmed to adjust a state of at least a portion of the mattress based on an identity of the individual on the mattress.

6. The smart bed of claim 5, further comprising:
an adjustable frame that carries the mattress and adjusts an orientation of at least the portion of the mattress in response to the identity of the individual on the mattress.

7. The smart bed of claim 5, further comprising:
a pressurization system that adjusts a pressure of at least the portion of the mattress in response to the identity of the individual on the mattress.

8. The smart bed of claim 5, further comprising:
a ventilation system that adjusts ventilation of at least the portion of the mattress in response to the identity of the individual on the mattress.

9. The smart bed of claim 5, further comprising:
a temperature control system that adjusts a temperature of at least the portion of the mattress in response to the identity of the individual on the mattress.

10. A smart bed, comprising:
a mattress including at least one sensor; and
a processor that communicates with the at least one sensor, the processor programmed to:
determine individual data comprising a respiratory rate, a heart rate, a temperature, a humidity, a sleeping location, and/or a sleeping position of an individual on the mattress;
process a combination of the individual data to identify the individual on the mattress; and
adjust at least one feature of the smart bed based on the identity of the individual on the mattress.

11. The smart bed of claim 10, wherein the at least one feature of the smart bed comprises:
an adjustable frame that carries the mattress and adjusts an orientation of at least the portion of the mattress in response to the identity of the individual on the mattress.

12. The smart bed of claim 10, wherein the at least one feature of the smart bed comprises:
a pressurization system that adjusts a pressure of at least the portion of the mattress in response to the identity of the individual on the mattress.

13. The smart bed of claim 10, wherein the at least one feature of the smart bed comprises:
a ventilation system that adjusts ventilation of at least the portion of the mattress in response to the identity of the individual on the mattress.

14. The smart bed of claim 10, wherein the at least one feature of the smart bed comprises:
a temperature control system that adjusts a temperature of at least the portion of the mattress in response to the identity of the individual on the mattress.

15. A method for adjusting at least one feature of a smart bed, comprising:
identifying an individual on a mattress of the smart bed; and
based on an identity of the individual on the mattress, adjusting the at least one feature of the smart bed based on a preference or a predetermined sleep condition of the individual.

16. The method of claim 15, wherein identifying the individual on the mattress comprises identifying the individual based on at least one of a respiratory rate, a heart rate, a humidity, a sleeping location, and/or a sleeping position of the individual on the mattress as determined with at least one sensor of the mattress.

17. The method of claim 15, wherein identifying the individual on the mattress comprises identifying the individual based on a combination of a respiratory rate, a heart rate, a humidity, a sleeping location, and/or a sleeping position of the individual on the mattress as determined with at least one sensor of the mattress.

18. The method of claim 15, wherein adjusting the at least one feature comprises at least one of:
adjusting an orientation of at least a portion of the mattress;
adjusting a pressure of at least the portion of the mattress;
actuating or adjusting ventilation of at least the portion of the mattress; and
adjusting a temperature of at least the portion of the mattress.

19. The method of claim 15, wherein adjusting the at least one feature comprises adjusting the at least one feature based on a predetermined preference of the individual on the mattress.

20. The method of claim 15, wherein adjusting the at least one feature comprises adjusting the at least one feature based on a predetermined sleep condition of the individual on the mattress.
